(19) United States
(12) Patent Application Publication
(10) Pub. No.: US 2012/0253130 A1
(43) Pub. Date: Oct. 4, 2012

(54) ENDOSCOPE HAVING EXTRA-FINE DIAMETER

(76) Inventor: Osamu Motoyama, Bunkyo-ku (JP)

(21) Appl. No.: 13/513,883

(22) PCT Filed: Dec. 21, 2010
(86) PCT No.: PCT/JP2010/072991

§ 371 Date: Jun. 5, 2012

(30) Foreign Application Priority Data
Dec. 22, 2009 (JP) 2009-290721

(51) Int. Cl. A61B 1/07

(52) U.S. Cl. 600/182

(57) ABSTRACT

Disclosed is an endoscope having an extra-fine diameter capable of improving strength and durability of outer and/or inner tube constituting an insertion portion of the endoscope while maintaining good insertion performance and operability, capable of easily and precisely locating lenses when various kinds of lenses are arranged. As such outer and/or inner tube constituting the insertion portion of the endoscope having an extra-fine diameter, a specific electrocast tube formed by a predetermined manufacturing method is used.

### FIELD OF THE INVENTION

The present invention relates to an endoscope having an extra-fine diameter to be used in medical or industrial purpose.

An endoscope having an extra-fine diameter for a medical purpose has been used by, for example, inserting a body of a patient to thereby observe an interior of the patient body. For an industrial purpose, the endoscope having an extra-fine diameter has been used for visual inspection of an interior of a fine tube or product.

The endoscope having the extra-fine diameter of such structure has a portion to be inserted (i.e., inserting or inserted portion, which is called “insertion portion” hereinlater), which is generally comprised of an outer tube as an exterior portion and one or more inner tube disposed inside the outer tube. Within the inner tube, an image guide fiber, an image sensor, a light guide fiber, or other various parts will be inserted.

Here, it is required for the insertion portion to be provided with no habit to be bent and to have good insertion ability or operability, and accordingly, various resin materials may be used as a material for forming the outer and inner tubes constituting the insertion portion (for example, refer to Patent Document 1).

Further, one or more various lenses are inserted to a front (distal) end portion of the insertion portion (for example, refer to Patent Document 2).

### PRIOR ART DOCUMENT

### Patent Document

Patent Document 1: Japanese Patent Laid-open Publication No. HEI 4-221525
Patent Document 2: Japanese Patent Laid-open Publication No. 2008-220710

### DESCRIPTION OF THE INVENTION

### Problems to be solved by the Invention

However, although various resins as a material for the outer and inner tubes constituting the insertion portion of an endoscope having an extra-fine diameter in addition to the material disclosed in the Patent Document 1 have been researched, in recent years, there is a tendency of making further small the diameter of the endoscope, which leads to a problem such that a resin is not suitable as a material in its strength. Particularly, in use for a dental purpose, since it is required for the insertion portion to be inserted into a hard teeth, a material having further strength and durability has been required, and hence, the problem mentioned above has further become conspicuous.

Then, although it has been partially performed to use metal material for a material of the outer tube and inner tube constituting the insertion portion in order to apply strength to the endoscope having an extra-fine diameter, in the case when these tubes are formed of the metal material, the tubes lack in flexibility, which may result in difficulty such that the insertion portion is not freely bent along a patient body portion or a product, and as a result, insertion performance or operability may often be damaged. Then, in order to solve such defective, use of meshed metal material has been considered, but in such case, the insertion portion has a large diameter, and an endoscope provided with such insertion portion is difficult for observation of an extra fine or narrow portion.

Furthermore, in the case where a resin material is adopted as a material forming the outer tube and inner tube constituting the insertion portion of the endoscope having an extra-fine diameter, it is necessary to arrange various kinds of lenses inside the outer and inner tubes. In such case, it is very difficult to locate a hard lens by accurately positioning it to a soft resin material. In order to realize such location, the use of other plurality of members such as jig has been required.

The present invention was made in consideration of the circumstances mentioned above, and an object of the present invention is to provide an endoscope having an extra-fine diameter capable of improving strength and durability of outer and inner tubes constituting an insertion portion of the endoscope while maintaining good insertion performance and operability, capable of easily and precisely locating lenses when various kinds of lenses are located inside the outer and inner tubes, and capable of freely adjusting an angle (so-called having flexibility) of a front end of the insertion portion in accordance with a shape or size of a portion to be observed.

### Means for Solving the Problem

A first invention conceived to solve the above problem provides an endoscope having an extra-fine diameter which includes an insertion portion formed, at a front end portion thereof, with an observation window and an illumination window, and in which an image guide fiber or an image sensor for transmitting an observation image taken into the insertion portion through the observation window, and a light guide fiber for transmitting an illumination light to the illumination window are arranged inside the insertion portion, wherein the insertion portion is provided with at least an outer tube as an exterior portion and one or more inner tube to be arranged inside the outer tube, either one or all of the outer and inner tubes are formed as electrocast tubes, the electrocast tube being one manufactured by: forming electrodeposit material or surrounding material, by an electroforming process, around a thin wire member formed, on an outer peripheral surface thereof, with a metal conductive layer composed of a substance different from that of the electrodeposit material or surrounding material; pulling one or both end portions of the thus formed thin wire member to deform the thin wire member so as to reduce a sectional area thereof to thereby form a clearance between the deformed thin wire member and the conductive layer; and pulling out and removing the deformed thin wire member from the electrocast tube with the conductive layer remaining inside the electrodeposit material or surrounding material, and the conductive layer has a conductivity higher than that of the electrodeposit material or surrounding material, and the electrocast tube after the thin wire material has been removed has an inner hollow portion having a circular or polygonal sectional shape.

A second invention conceived to solve the above problem provides an endoscope having an extra-fine diameter which includes an insertion portion formed, at a front end portion thereof, with an observation window and an illumination window and in which an image guide fiber or an image sensor for transmitting an observation image taken into the insertion portion through the observation window and a light guide fiber for transmitting an illumination light to the illumination window are arranged inside the insertion portion, wherein the insertion portion is provided with at least an outer tube as an exterior portion and one or more inner tube to be arranged inside the outer tube, and either one or all of the outer and inner tubes is formed as an electrocast tube, the electrocast tube being an ultra-fine Ni electrocast tube having a polycrystalline structure composed of fine crystal grains each having a grain diameter of 5 nm to 300 nm, an elasticity of Young's modulus of 93 GPa to 191 GPa, and a hardness of Vickers hardness of 300 to 600.

Furthermore, in the above first and second inventions, it may be desired that a tapered tube is provided at a front end portion of the image guide fiber or the image sensor so as to extend toward the observation window, and the tapered tube is formed by forming a second electrocast tube by effecting a secondary electroforming process to a first electrocast tube which is mounted with a core wire which is electroformed so that a part thereof is exposed, and then, pulling out the core wire and the first electrocast tube from the second cast tube.

In the above invention, at least one lens may be arranged inside the tapered tube.

Furthermore, in the above invention, it may be desired that the tapered tube and the image guide fiber or image sensor are integrated as an integrated body in such a manner that a distance between the integrated body and the observation window is adjustable.

Still furthermore, in the above first and second inventions, it may be desired that one of the inner tube is formed as a tapered tube tapered toward the observation window, the tapered tube is formed by forming a second electrocast tube by effecting a secondary electroforming process to a first electrocast tube which is mounted with a core wire which is electroformed so that a part thereof is exposed, and then, pulling out the core wire and the first electrocast tube from the second cast tube, and the image guide fiber or image sensor is inserted into the tapered tube to be disposed therein.

In the above case, at least one lens may be arranged inside the tapered tube.

Furthermore, it may be desired that another lens is located at a front end portion of the image guide fiber or image sensor, and by automatically inserting the image guide fiber or image sensor provided with the lens into or out of the tapered tube, the distance between the lens arranged inside the tapered tube and the lens provided at the front end portion of the image guide fiber or image sensor becomes freely adjustable.

It may further be desired that another inner tube is disposed inside the tapered tube, and the image guide fiber or image sensor is inserted into the another inner tube to be disposed therein.

### Effects of the Invention

According to the endoscope having an extra-fine diameter of the above-described first and second invention, since the electrocast tube formed by a predetermined manufacturing method is adopted as the outer and/or inner tube constituting the insertion portion of the endoscope, it becomes possible to further improve strength and durability of the electrocast tube in comparison with a resin tube, and in a dental technical filed, the endoscope is preferably usable. In addition, in the electrocast tube adopted for the endoscope having an extra-fine diameter of the present invention, since the electrocast tube has a thickness which is freely designable and has elasticity, there is no fear of causing a problem concerning flexibility which was raised in a case of using a conventional metal tube. Furthermore, the electrocast tube can be formed with an outer diameter of less than 1 mm, thus satisfying the recent requirement for providing a fine-diameter tube. Still furthermore, according to the present invention, the tube constituting the insertion portion of the endoscope having an extra-fine diameter is formed of a predetermined electrocast tube, it is possible to preliminarily deform the insertion portion so as to accord with the shape of an observation portion (that is, possible to preliminarily bent the insertion portion with a predetermined angle), a more fine portion can be precisely observed.

Furthermore, in the endoscope having an extra-fine diameter according to the first and second inventions, since the tapered tube formed by a predetermined method is provided at the front end portion of the image guide fiber or image sensor, the tapered tube can function as so-called “throttle”, a clear and fresh image can be obtained without designing a complicated lens for the front end portion of the insertion portion. Still furthermore, since the predetermined manufacturing method for forming the tapered tube has high manufacturing accuracy, the diameter of the front end portion of the tapered tube having an extra-fine diameter functioning as “throttle” can be optionally designed. Further, the use of the tapered tube as an inner tube into which the image guide fiber or like is inserted may achieve substantially the similar effects to those mentioned above.

Still furthermore, in the endoscope having an extra-fine diameter, by mounting a lens inside the tapered tube, various effects in addition to the mere “throttle” function can be achieved. Moreover, as mentioned above, since the predetermined manufacturing method for forming the tapered tube has high accuracy, the inner diameter thereof can be optionally designed, and in addition, the diameter of the lens to be arranged inside the tapered tube can also be optionally designed and the “throttle” of the front end portion of the tapered tube and the distance between the tapered tube and the lens arranged therein can be optionally and precisely designed.

Still furthermore, the tapered tube adopted for the endoscope having an extra-fine diameter of the present invention is not made of resin, and formed as the electrocast tube having high performance, so that the lens can be mounted with high yield and at highly precisely.

Still furthermore, in the endoscope having an extra-fine diameter described above, it is possible to integrally form the tapered tube and the image guide fiber or image sensor as an integrated body and to freely adjust the distance between the integrated body and the observation window, and a so-called “focusing” function can be added.

In the meantime, such “focusing” function may be added by using the tapered tube as an inner tube for the image guide fiber or image sensor, disposing the lens to the front end portion of this image guide fiber or like, and manually or automatically inserting the image guide fiber or like into or out of the tapered tube.

Still furthermore, the “focusing” function may be also added by arranging another inner tube inside the tapered tube, inserting and arranging the image guide fiber or image sensor inside this another tube, and relatively moving the positional relationship between the tapered tube and the inner tube disposed inside the tapered tube (that is, by sliding the inner tube inside the tapered tube).

### BRIEF DESCRIPTION OF THE DRAWINGS

FIG. 1 is a schematic sectional view showing an insertion portion (specifically, front end portion thereof) of an endoscope having an extra-fine diameter according to one embodiment of the present invention.

FIG. 2 includes (a) and (b) both showing schematic sectional views of an insertion portion (specifically, front end portion thereof) of an endoscope having an extra-fine diameter according to another embodiment of the present invention.

FIG. 3 includes (a) and (b) both showing schematic sectional views of an insertion portion (specifically, front end portion thereof) of an endoscope having an extra-fine diameter according to a further embodiment of the present invention.

FIG. 4 is a schematic sectional view of an insertion portion (specifically, front end portion thereof) of an endoscope having an extra-fine diameter according to a further embodiment of the present invention.

FIG. 5 is a schematic sectional view of an insertion portion (specifically, front end portion thereof) of an endoscope having an extra-fine diameter according to a further embodiment of the present invention.

FIG. 6 is a schematic sectional view of an insertion portion (specifically, front end portion thereof) of an endoscope having an extra-fine diameter according to a further embodiment of the present invention.

### MODE FOR CARRYING OUT THE INVENTION

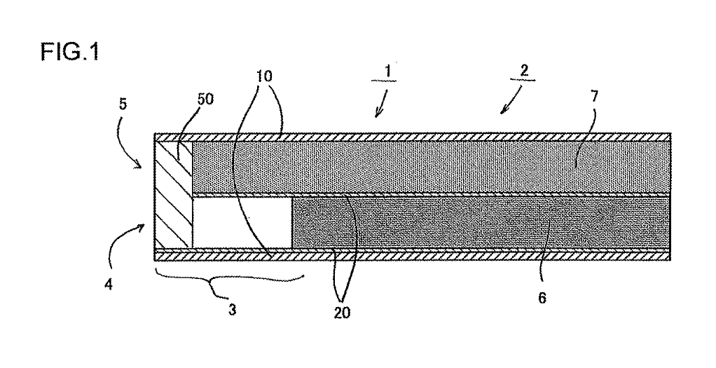

FIG. 1 is a schematic sectional views an insertion portion (specifically, front end portion thereof) of an endoscope having an extra-fine diameter according to one embodiment of the present invention.

As shown in FIG. 1, an endoscope 1 having an extra-fine diameter according to the present invention has an insertion portion 2 (which may be substituted with an inserting portion or inserted portion) provided with an observation window 4 and an illumination window 5 at a front end portion thereof. In addition, the endoscope 1 is provided with an image guide fiber 6 for transmitting an observed image taken through the observation window 4 and a light guide fiber 7 for transmitting illumination light to the illumination window 5, both the fibers 6 and 7 being inserted and disposed into the insertion portion 2.

Further, with reference to this figure, a cover member 50 is mounted to the front end portion of the endoscope 1 having the extra-fine diameter so as to protect the observation window 4 and the illumination window 5, which however may be optionally provided. Furthermore, in the following explanation, as shown in the figures, although the description is made with reference to an example of a case in which an image guide fiber 6 is used as means for transmitting an observed image taken from the observation window 4, the present embodiment is not limited to this example, and an image sensor may be used in place of the image guide fiber 6. Such image sensor means a photoelectric conversion element formed as an integrated circuit by utilizing a semiconductor manufacturing technology, and more specifically, means a CCD image sensor, CMOS image sensor, or like. These sensors may be utilized in the present invention in place of the image guide fiber 6.

The insertion portion 2 of the endoscope 1 having the extra-fine diameter according to the present invention includes an outer tube 10 as an outer configuration and one or more inner tubes (inner tube 20 for the image guide fiber in FIG. 1) to be located inside the outer tube 10, and the endoscope 1 of the present invention is characterized by forming the insertion portion 2 in which either one or both of the outer tube 10 and inner tube 20 are composed of a specific electrocast tube formed by a predetermined manufacturing method.

In the followings, although description is made with respect to an example of an image guide fiber as an inner tube, the present invention is not limited to such example, and as far as the insertion portion is provided with an inner tube adopted for light guide, various kinds of inner tubes may be used by being formed as a specific electrocast tube formed by a predetermined manufacturing method, which will be explained hereinafter.

Furthermore, it is permissible for the present invention to use a predetermined electrocast tube for at least either one of the outer and inner tubes constituting the endoscope having the extra-fine diameter. Accordingly, in a case where a predetermined electrocast tube is used as an outer tube, for example, any kind of tube may be used as an inner tube, and in such case, an inner tube may be formed with a cover resin covering an entire periphery of a bundle of a plurality of image guide fibers. Otherwise, in a case where a resin tube is used as an outer tube, for example, the endoscope having an extra-fine diameter according to the present invention will be provided by using a predetermined electrocast tube as an inner tube (as inner tube 20 for image guide fiber 6).

Herein, an electrocast tube used for the outer tube and/or inner tube 20 for image guide fiber constituting the insertion portion of the endoscope having an extra-fine diameter according to the present invention will be explained.

The electrocuting tubes used for the present invention are the following two tubes (which may be called “electrocast tube A” and “electrocast tube B”).

(Electrocast Tube A)

The electrocast tube A is formed by: forming an electrodeposit material or surrounding material by an electroforming process around a thin wire member provided, on an outer peripheral surface thereof, with a metal conductive layer of a substance different from the electrodeposit material or surrounding material; deforming the thus formed thin wire member so as to make small a sectional area by pulling one or both ends thereof; and forming a clearance between the deformed thin wire member and the conductive layer and pulling out the deformed thin wire member so as to remove the thin wire member with the conductive layer remaining inside the electrodeposit material or surrounding material. In such electrocast tube A, the conductive layer has an electrical conductivity higher than that of the electrodeposit material or surrounding material, and a hollow portion formed by removing the thin wire member has an inner shape having a circular section or polygonal section.

(Electrocast Tube B)

The electrocast tube is an Ni electrocast ultra-fine tube which is characterized by having a polycrystalline body composed of fine crystal grains each having a grain diameter of 5 nm to 300 nm, an elasticity of Young's modulus of 93 GPa to 191 GPa, and a hardness of Vickers harness of 300 to 600.

Herein, the electrocast tubes A and B are both manufactured by LUZCOM INC., and more specifically, the electrocast tube A is one disclosed in Japanese Patent No. 3889689 to LUZCOM INC. and the electrocast tube B is one disclosed in International Laid-open Publication No. WO2006/135057.

The inventor of the subject application has always searched for an endoscope, and in particular, for an endoscope having an extra-fine diameter preferably usable for a dental technical field, and during the search, the inventor found out the presence of the electrocast tubes A and B of LUZCOM INC., and conceived the application of these tubes as an outer tube and/or inner tube for image guide fiber of an endoscope, and thus, the present invention was completed. More specifically, the inventor paid his attention to the fact that the electrocast tubes A and B have appropriate strength and durability regardless of having extremely fine diameter, these electrocast tubes A and B can be preliminarily changed in their angles in accordance with a shape of a portion to be observed (i.e., can be preliminarily bent at a predetermined angle), and the tubes A and B have high dimensional performance, i.e., hence, having high performance for attaching lenses. According to such finding, the inventor completed the present invention by adopting such electrocast tubes A and B specifically to an endoscope having an extra-fine diameter for the use of dental technical field.

Next, another embodiment of the present invention will be also described hereunder with reference to accompanying drawings.

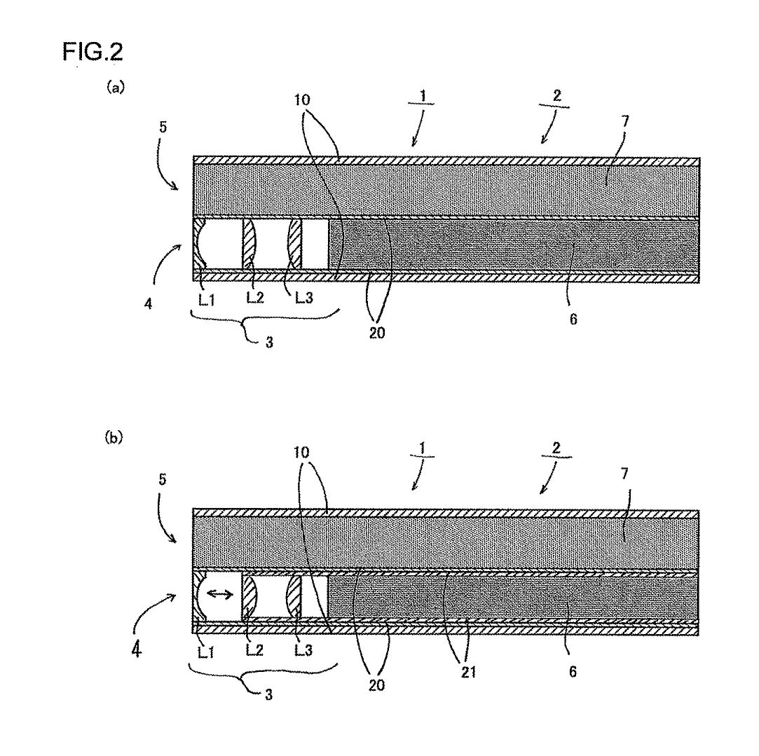

FIG. 2 is a schematic sectional view of an insertion portion (front end portion) of an endoscope 1 having an extra-fine diameter according to another embodiment of the present invention.

As shown in FIG. 2(a), in the endoscope having the extra-fine diameter of the present embodiment, various kinds of lenses L (L1, L2, L3) may be arranged in front of the image guide fiber 6, and herein, the present embodiment does not specifically limit kinds or numbers of the lenses, which are optionally designed in accordance with the use or like thereof.

For example, as shown in FIG. 2, a concave lens L1 is disposed inside the inner tube 20 for image guide fiber at the most distal end formed as the observation window 4, and convex lenses L2 and L3, opposing to each other, disposed inside the inner tube 20 for image guide fiber at positions between the most distal end of the image guide fiber 6 and the concave lens L1. Further, it may be possible to use SELFOC lenses in place of the concave and convex lenses.

At a time when such various kinds of lenses are disposed, in the present invention, as described above, since the specific electrocast tube is used as the inner tube 20 for the image guide fiber, the preferred dimensional performance can be achieved, and in addition, since the electrocast tube having a certain rigidity is used, the lenses can be easily set and positioned.

Furthermore, as shown in FIG. 2(b), in an arrangement in which the convex lenses L2, L3, and the image guide fiber 6 are disposed in assembly within the inner tube 21, and then, the distance between the convex lenses L2, L3 and the concave lens L1 can be adjusted by sliding the inner tube 21 within the inner tube 20 in an double-arrowed direction, thus endowing “focusing function” to the endoscope 1.

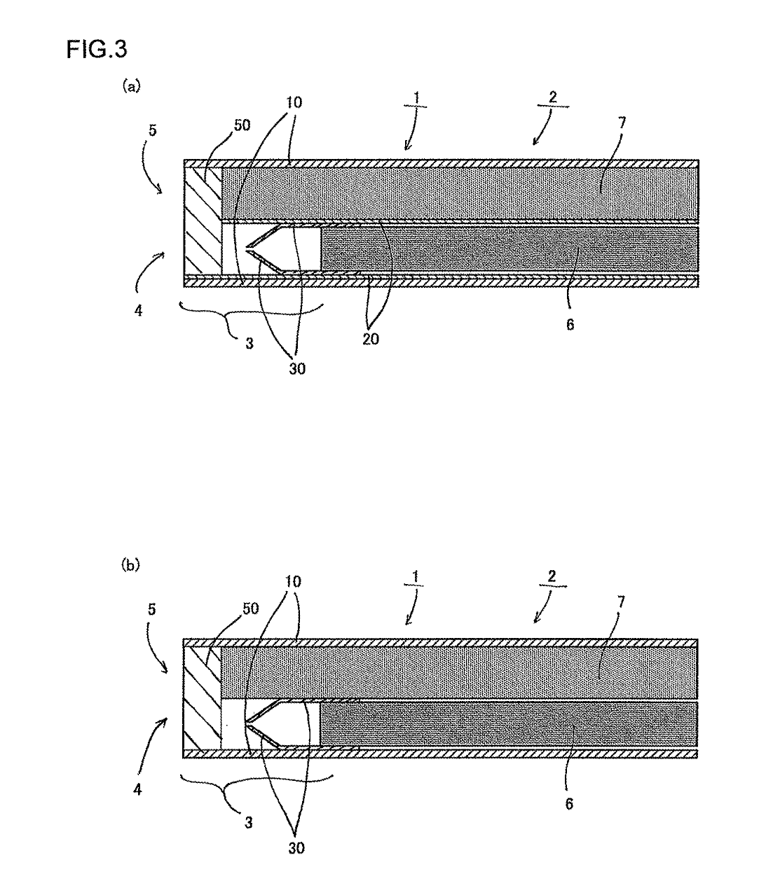

FIG. 3 is also a schematic sectional view of an insertion portion (front end portion) of an endoscope having an extra-fine diameter according to a further embodiment of the present invention.

As shown in FIG. 3(a), in the endoscope 1 having the extra-fine diameter of the present embodiment, a tapered tube 30 tapered from the front end of the image guide fiber 6 toward the observation window 5 is disposed to the front end portion of the image guide fiber 6, and the tapered tube 30 is an electrocast tube manufactured by a predetermined manufacturing method.

Furthermore, in the arrangement of FIG. 3(a), as like as in FIGS. 1 and 2, an inner tube 20 formed of an electrocast tube or a “tube” made of another substance is disposed, but the present embodiment is not limited to this embodiment, and such inner tube may be substituted with a coat resin covering the tapered tube 30 and the image guide fiber 6 with no presence of the inner tube formed as the electrocast tube.

Hereunder, an electrocast tube used as the tapered tube 30 will be first explained.

The electrocast tube utilised for the present embodiment has characters such as follows (which may be called “electrocast tube C” hereinafter).

(Electrocast Tube C)

An electroforming process was performed so that a part of a core line is exposed. A second electrocast tube was applied to a first electrocast tube with the core line being attached, thus forming a second electrocast tube. The tapered tube was then formed by pulling the core line and the first electrocast tube out of the second electrocast tube.

The electrocast tube C is an electrocast tube manufactured by LUZCOM INC., and more specifically, the electrocast tube disclosed in Japanese Patent Laid-open Publication No. 2006-233244 of LUZCOM INC.

The inventor of the subject application found out the electrocast tube C of LIZCOM INC. as well as electrocast tubes A and B and conceived the use as an inner tube of an endoscope having an extra-fine diameter to thereby apply a function as “throttle” such as pin-hole of a camera to the tapered portion of the inner tube, thus completing the present invention. According to the present invention, it becomes possible to achieve the “throttling” function and obtain more sharp and clean image without using complicated lens arrangement and a plurality of parts or members.

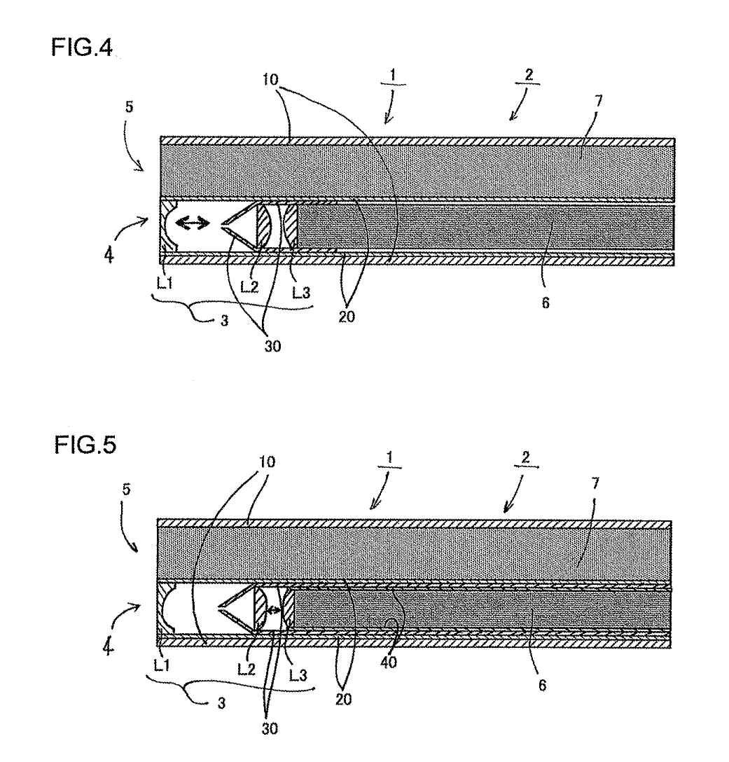

FIG. 4 is also a schematic sectional view of an insertion portion (specifically, front end portion thereof) of an endoscope having an extra-fine diameter according to a further embodiment of the present invention.

As described above, even in a case where the tapered tube (i.e., electrocast tube C) is used as an inner tube, as like as the embodiment of FIG. 2, various kinds of lenses L may be arranged in front of the image guide fiber 6. For example, as shown in FIG. 4, the concave lens L1 is arranged to the most distal end portion as the observation window 4, and the convex lenses L2 and L3 facing to each other may be arranged inside the tapered tube 30 positioned at the front end portion of the image guide fiber 6.

Moreover, in an embodiment in which the tapered tube 30 is used for the endoscope having an extra-fine diameter of the present invention, the tapered tube 30 and the image guide fiber 6 may be formed to be integrated in a manner such that the distance between the integrated assembly and the observation window 4 is freely adjustable (that is, the tapered tube 30 and the inner tube 20 for the image guide fiber are moved as an integrated assembly in the arrowed direction shown in FIG. 4).

According to the adoption of such structure as mentioned above, “focusing (focus slide) function” can be applied in addition to the “throttling function”.

FIG. 5 is also a schematic sectional view of an insertion portion (specifically, front end portion thereof) of an endoscope having an extra-fine diameter according to a further embodiment of the present invention.

As described above, in the case where the tapered tube (i.e., electrocast tube C) is used as an inner tube, it may be possible to provide the embodiment shown in FIG. 5. That is, another inner tube 40 formed from either one of the electrocast tubes A and B is disposed inside the tapered tube 30 so as to be utilized as an inner tube for the image guide fiber. In this embodiment, it may be possible to arrange various kinds of lenses L in front of the image guide fiber as like as the embodiment shown in FIG. 4. For example, as shown in FIG. 5, it may be permissible to dispose the concave lens L1 to the most front end portion forming the observation window 4, dispose the convex lens L3 to the front end portion of the image guide fiber 6 inside the inner tube 40 for the image guide fiber, and dispose the lens L2 inside the tapered tube 30 so as to face the convex lens L3. It is further to be noted that, in the embodiment of FIG. 5, it is not always necessary to dispose the inner tube 20, which may be optionally disposed.

According to the configuration mentioned above, it is possible to slide the inner tube 40 for the image guide fiber inside the tapered tube 30 (along the direction of the double-arrow in FIG. 5), and in addition, the focusing function as well as the throttling function can be applied as like as the former embodiment.

Furthermore, as shown in FIGS. 2. 4 and 5, in the case where the various kinds of lenses are disposed to the front end portion of the endoscope having an extra-fine diameter, it may take an arrangement in which the thickness of the outer tube or inner tube (particularly, an inner tube for the image guide tube among the inner tubes) positioned to the portion at which the lens is disposed, is made thickened or only the tube positioned to that portion is constructed so as to provide a multiple-structure. By adopting the arrangement mentioned above, the rigidity of the portion 3 can be increased and make optically stable.

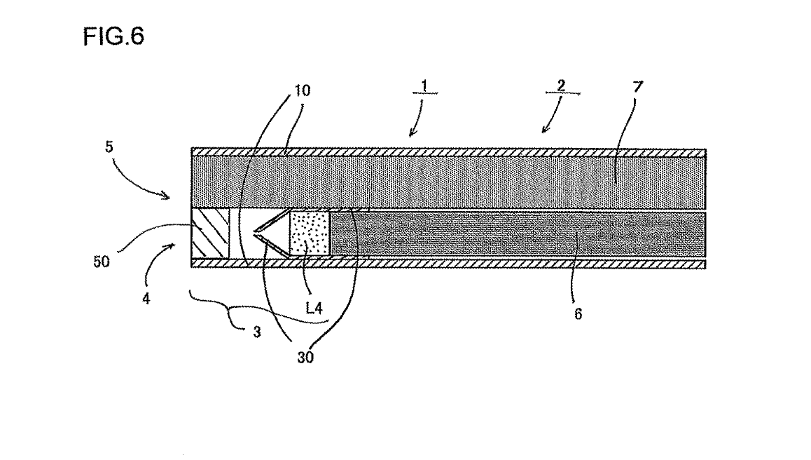

FIG. 6 is also a schematic sectional view of an insertion portion (specifically, front end portion thereof) of an endoscope having an extra-fine diameter according to a further embodiment of the present invention.

As described hereinbefore, in the case of using the tapered tube (electrocast tube C as an inner tube), an embodiment represented by FIG. 6 may be adopted. That is, a cover member 50 for protecting the observation window 4 may be provided and the SELFOC lens L4 may be disposed within the tapered tube 30.

According to the adoption of such arrangement, a very simple lens structure can be realized. Further, in such embodiment, as shown in FIG. 4 or 5, the focusing function may be applied by sliding the tapered tube 30 and the like.

Furthermore, it is to be noted that parts or members constituting the endoscope having an extra-fine diameter other than those explained hereinabove are not specifically limited in the present invention, and conventionally known various parts or members may be optionally used.

Still furthermore, in a case where there is a fear that substance to be used as electrocast tubes A, B and/or C adversely may affect on a human body, Au plating, Pt plating or Ag plating may be effected on the surface of the electrocast tubes (particularly, the electrocast tube C used as the outer tube 10).

### EXPLANATION OF REFERENCE NUMERALS

1—endoscope having extra-fine diameter
2—insertion portion
3—front end portion
4—observation window
5—illumination window
6—image guide fiber
7—light guide fiber
10—outer tube
20, 21, 40—inner tube for image guide fiber
30—tapered tube
50—cover member
L—lens

1. An endoscope having an extra-fine diameter which includes an insertion portion formed at a front end portion thereof with an observation window and an illumination window, and in which an image guide fiber or an image sensor for transmitting an observation image taken into the insertion portion through the observation window and a light guide fiber for transmitting an illumination light to the illumination window are arranged inside the insertion portion, wherein
the insertion portion is provided with at least an outer tube as an exterior portion and one or more inner tube to be arranged inside the outer tube,
either one or all of the outer and inner tubes are formed as electrocast tubes, the electrocast tube being one manufactured by: forming electrodeposit material or surrounding material, by an electrocast process, around a thin wire member formed, on an outer peripheral surface thereof, with a metal conductive layer composed of a substance different from that of the electrodeposit material or surrounding material; pulling one or both end portions of the thus formed thin wire member to deform the thin wire member so as to reduce a sectional area thereof to thereby form a clearance between the deformed thin wire member and the conductive layer; and pulling out and removing the deformed thin wire member from the electrocast tube with the conductive layer remaining inside the electrodeposit material or surrounding material, and the conductive layer has a conductivity higher than that of the electrodeposit material or surrounding material, and the electrocast tube after the thin wire material has been removed has an inner hollow portion having a circular or polygonal sectional shape.

2. An endoscope having an extra-fine diameter which includes an insertion portion formed at a front end portion thereof with an observation window and an illumination window, and in which an image guide fiber or an image sensor for transmitting an observation image taken into the insertion portion through the observation window and a light guide fiber for transmitting an illumination light to the illumination window are arranged inside the insertion portion, wherein
the insertion portion is provided with at least an outer tube as an exterior portion and one or more inner tube to be arranged inside the outer tube, and either one or all of the outer and inner tubes is formed as an electrocast tube, the electrocast tube being an ultra-fine Ni electrocast tube having a polycrystalline structure composed of fine crystal grains each having a grain diameter of 5 nm to 300 nm, an elasticity of Young's modulus of 93 GPa to 191 GPa, and a hardness of Vickers hardness of 300 to 600.

3. The endoscope having an extra-fine diameter according to claim 1, wherein a tapered tube is provided at a front end portion of the image guide fiber or the image sensor so as to extend toward the observation window, and the tapered tube is formed by forming a second electrocast tube by effecting a secondary electroforming process to a first electrocast tube which is mounted with a core wire which is electroformed so that a part thereof is exposed, and then, pulling out the core wire and the first electrocast tube from the second cast tube.

4. The endoscope having an extra-fine diameter according to claim 3, wherein at least one lens is arranged inside the tapered tube.

5. The endoscope having an extra-fine diameter according to claim 4, wherein the tapered tube and the image guide fiber or image sensor are integrated as an integrated body in such a manner that a distance between the integrated body and the observation window is adjustable.

6. The endoscope having an extra-fine diameter according to claim 1, wherein one of the inner tube is a tapered tube tapered toward the observation window, the tapered tube is formed by forming a second electrocast tube by effecting a secondary electroforming process to a first electrocast tube which is mounted with a core wire which is electroformed so that a part thereof is exposed, and then, pulling out the core wire and the first electrocast tube from the second cast tube, and the image guide fiber or image sensor is inserted into the tapered tube to be disposed therein.

7. The endoscope having an extra-fine diameter according to claim 6, wherein at least one lens is arranged inside the tapered tube.

8. The endoscope having an extra-fine diameter according to claim 7, wherein another lens is provided at a front end portion of the image guide fiber or image sensor, and by automatically inserting the image guide fiber or image sensor provided with the lens into or out of the tapered tube, the distance between the lens arranged inside the tapered tube and the lens provided at the front end portion of the image guide fiber or image sensor becomes freely adjustable.

9. The endoscope having an extra-fine diameter according to claim 8, wherein another inner tube is disposed inside the tapered tube, and the image guide fiber or image sensor is inserted into the another inner tube to be disposed therein.

10. The endoscope having an extra-fine diameter according to claim 2, wherein a tapered tube is provided at a front end portion of the image guide fiber or the image sensor so as to extend toward the observation window, and the tapered tube is formed by forming a second electrocast tube by effecting a secondary electroforming process to a first electrocast tube which is mounted with a core wire which is electroformed so that a part thereof is exposed, and then, pulling out the core wire and the first electrocast tube from the second cast tube.

11. The endoscope having an extra-fine diameter according to claim 2, wherein one of the inner tube is a tapered tube tapered toward the observation window, the tapered tube is formed by forming a second electrocast tube by effecting a secondary electroforming process to a first electrocast tube which is mounted with a core wire which is electroformed so that a part thereof is exposed, and then, pulling out the core wire and the first electrocast tube from the second cast tube, and the image guide fiber or image sensor is inserted into the tapered tube to be disposed therein.
